(19) United States
(12) Patent Application Publication
(10) Pub. No.: US 2009/0017486 A1
(43) Pub. Date: Jan. 15, 2009

(54) METHOD AND TEST KIT FOR THE IDENTIFICATION OF COMPOUNDS SPECIFICALLY MODULATING THE ACTION OF CORTICAL AXO-AXONIC CELLS

(76) Inventors: Gabor Tamas, Szeged (HU); Pal Barzo, Szeged (HU)

Correspondence Address: HAHN & VOIGHT PLLC, 1012 14TH STREET, NW, SUITE 620, WASHINGTON, DC, 20005, US

(21) Appl. No.: 12/088,257

(22) PCT Filed: Sep. 29, 2006
(86) PCT No.: PCT/IB2006/053557

§ 371 Date: Jul. 9, 2008

(30) Foreign Application Priority Data
Sep. 30, 2005 (HU) P0500908

(51) Int. Cl. C12Q 1/02

(52) U.S. Cl. 435/29

(57) ABSTRACT

The present invention provides a method for identifying an agent capable of specifically modulating or eliminating the action of axo-axonic cells, an experimental kit for performing the said method and compounds identifiable by the said method, as well as methods for the treatment or prevention different neurological symptoms by modulating the activity of axo-axonic cells.

### TECHNICAL FIELD

The present invention provides a method for identifying an agent capable of specifically modulating or eliminating the action of axo-axonic cells, an experimental kit for performing the said method and compounds identifiable by the said method, as well as methods for the treatment or prevention different neurological symptoms by modulating the activity of axo-axonic cells.

### BACKGROUND ART

The axon has the lowest threshold for action potential generation of the various subcellular compartments in cortical neurons [Stuart et. al. (1997); Colbert and Pan (2002)]. Axons in the cerebral cortex receive synaptic input only at the axon initial segment almost exclusively from chandelier or axo-axonic cells (AACs) [Szentagothai and Arbib (1974); Somogyi (1977)], which use the dominant inhibitory neuro-transmitter, gamma-aminobutyric acid (GABA) [Freund and Buzsaki (1996); Somogyi et al. (1998)]. Thus, axo-axonic cells are considered as strategically placed inhibitory neurons controlling action potential generation and cortical information flow [Freund and Buzsaki (1996); Somogyi at al. (1998)].

Intracellular chloride concentrations ([Cl⁻]ᵢₙ) and, as a consequence, the polarity of GABAergic responses is predominantly determined by chloride extrusion mechanisms [Misgeld et al. (1986); Staley et al. (1995); Payne et al. (2003)]. In the mature cerebral cortex, the K⁺—Cl⁻ cotransporter 2 (KCC2) is responsible for the generation of an inwardly directed Cl⁻ electrochemical gradient rendering GABA hyperpolarizing [Rivera et al. (1999)]. Differential KCC2 expression in distinct types of neurons was shown to influence reversal potentials in various brain regions [Martina et al. (2001); Gulacsi et al. (2003); Gavrikov et al. (2003); Chavas and Marty (2003)], but quantification of this cotransporter in different regions of neurons is lacking.

The relative timing of spikes, spike to spike coupling and synchronization is involved in cognitive processes [Singer and Gray (1995)], thus the powerful excitatory action of axo-axonic cells mimicked by an agent or compound might effectively influence learning and memory and spike time dependent plasticity [Dan and Poo (2004)], perceptual grouping [Singer and Gray (1995)], and several mental conditions such as schizophrenia [Lewis et al. (2005)] and epilepsy [Arellano et al. (2004)] in which the function of axo-axonic cells is thought to be crucial [Howard et al. (2005)].

It is clear from the above that there is a need in the art for providing means to generate site-specific and selective modulation of cortical axo-axonic cells and thus develop specific agents to influence the course of several important neural processes. However, the art definitely lacks selective agents to investigate these processes and thus the possibilities for developing specific agents and possible future therapeutics and therapies are severely limited.

The present inventors surprisingly found that single spikes in axo-axonic cells can trigger action potentials in pyramidal cells and initiate stereotyped series of multiple synaptic events in the cortical network. The unexpected reliability of GABAergic, axo-axonal spike triggering in pyramidal cells can be similar to that of unitary monosynaptic transmission. The excitatory action of axo-axonic cells is based on a de-polarized reversal potential for axonal relative to perisomatic GABAergic inputs, which is supported by the absence of the potassium-chloride co-transporter 2 (KCC2) from the axonal membrane. Powerful spike transmission from axo-axonic to pyramidal cells provides a novel mechanism for reliable signal propagation and grouping in cortical networks.

With respect to the present specification and claims, the foregoing technical terms will be used in accordance with the below given definitions. With regard to the interpretation of the present invention, it shall be understood that the below defined terms are used in accordance with the given definitions even if said definitions might not be in perfect harmony with the usual interpretation of said technical term.

In the context of the present invention “axo-axonic cell” is defined as a cell type in the brain giving rise to characteristic axonal cartridges (vertical rows of axon terminals) and target the axon initial segment of postsynaptic neurons. The alternative name for the same type of cell in the literature is chandelier cell [Szentagothai and Arbib (1974), Somogyi (1977), Howard et al. (2005)].

As used herein, “EPSP” or “excitatory postsynaptic potential” means a change of the postsynaptic membrane potential by the action of a synaptically released neuro-transmitter making the postsynaptic neuron more likely to fire action potentials.

As used herein, “IPSP” or “inhibitory postsynaptic potential” means a change of the postsynaptic membrane potential by the action of a synaptically released neuro-transmitter making the postsynaptic neuron less likely to fire action potentials.

As used herein, the term “stimulus” is a signal that directly influences the activity of a living organism or one of its parts (e.g. nerve cell). In the particular case where the stimulus is applied to a nerve cell, a stimulus is defined in it widest meaning and can be in any of various different forms including, but not limited to electrical signals, light, electromagnetic and/or magnetic fields, ions, transmitters, drugs, other compounds, intercellular connections, especially synapse of any type.

“Action” of neuron is the effect of the activity of one neuron on the target neuron or neurons.

“Response” of any given cell is the activity or inhibition of previous activity of an organism or any of its parts resulting from stimulation.

In the context of the present invention an “activator” or “stimulator” or “stimulating agent” is an agent or substance that increases the activity of an organism or any of its parts.

In the context of the present invention a “blocker”, “blocking agent” or “inhibitor” is an agent or substance that decreases or terminates the activity of an organism or any of its parts.

As used herein, a “modulator” is an agent or substance that changes the characteristics of activity of an organism or any of its parts.

As used herein, the term “firing characteristics” includes the waveform, temporal pattern, frequency, threshold and ionic composition of action potentials. Firing characteristics is “enhanced” provided that the firing rate increases, the number of action potentials is higher in a given pattern, the waveform of an action potential is lengthened, and/or action potential threshold is decreased. Firing characteristics is “decreased” provided that the firing rate decreased, the number of action potentials is smaller in a given pattern, the waveform of an action potential is shortened and/or the action potential threshold is increased.

### DISCLOSURE OF INVENTION

The present invention provides a method for identifying an agent capable of specifically modulating or eliminating the action of axo-axonic cells, comprising the steps of

a) identifying an axo-axonic cell connected to the initial segment of the axon (within 30-40 μM from the soma or within the region situated between the soma and the first branchpoint of the axon) of at least one functional postsynaptic pyramid cell in a biological sample, cell culture or live animal,

b) stimulating the said axo-axonic cell,

c) measuring the reversal of postsynaptic potential evoked by the said axo-axonic cell,

d) exposing the said axo-axonic cell to a test compound,

e) repeating step b),

f) repeating step c),

g) identifying the test compound as a specific modulator or blocking agent of axo-axonic cells if the measured reversal of postsynaptic potential in step f) significantly differs from that measured in step c).

The method of the present invention comprises the identification of synaptic connections of an AAC to be stimulated by the putative stimulating agent. Although several examples are given in the present specification, these connections and networks are not limited to those exemplified. In general, AACs were determined to induce postsynaptic potential change in several settings, with several different outcome, i.e. monosynaptic and disynaptic connections producing depolarizing, subthreshold de-polarizing or long latency depolarizing postsynaptic potentials.

The AACs can be exposed to the putative stimulating agent using techniques well known in the art. This procedure is highly dependent on the experimental setup used, but it can be carried out by a person skilled in the art without any difficulty using the present disclosure. The step of exposing the test compound to the AAC may involve contacting a solution of the test compound with the cell, or even through the identification of a presynaptic cell, and contacting the test compound with that presynaptic cell and stimulating thereby the AAC through a synapse.

In preferred embodiments of the present invention, test compounds can be screened for their ability to stimulate specifically evoked GABAergic depolarizing postsynaptic potentials. This screening procedure can be targeted to a variety of compounds, including but not limited to testing select compounds, testing known compounds for their additional actions for stimulating AACs, testing members of chemical libraries of different size, and the like. The screening according to the invention may also be accomplished using several techniques well known in the art, for example in vitro on cell or tissue cultures, ex vivo on tissue sections or isolated organs, or in vivo in a test animal.

It will be apparent to a person skilled in the art that the concentration of the test compound may vary widely to achieve the stimulation of AACs. The techniques necessary to optimize the test conditions are well within the skill of the art. The effect of the test compound is measured through the response it generates on the postsynaptic cell connected to the AAC. There are several standard techniques to measure the induction of depolarizing postsynaptic potentials in the postsynaptic cell, such as patch clamp or intracellular recordings in current clamp or voltage clamp mode, recording optical signals intrinsic to the tissue and imaging signals of voltage sensitive and ion concentration selective compounds. In case the depolarizing postsynaptic potential is identified, the test compound is identified as a stimulating agent of AAC according to the invention.

In another embodiment, the present invention provides an experimental kit for performing the method according to the invention comprising the following:
a cell culture comprising axo-axonic cells,
a medium comprising instructions—and/or optionally means—for performing the method according to the invention,

and optionally further comprising
at least one postsynaptic cell connected to an axo-axonic cells,
means and/or instructions for detecting depolarizing postsynaptic potential on the postsynaptic pyramid cell evoked by the said axo-axonic cells,
appropriate buffers and culture media.

In the context of this aspect of the present invention, standard cell or tissue culture techniques are used for the preparation of either homogeneous or heterogeneous nerve cell cultures. Such techniques are known to the person skilled in the art. Specific methods optimized for culturing axo-axonic cells are used or alternative procedures can be modified to generate the cell culture for the purposes of the present invention. Non-limiting examples include FR2822474, RU2191388, U.S. Pat. No. 5,721,139 and EP0776968.

The response can be detected in a variety of ways, such as by using patch clamp or intracellular recordings in current clamp or voltage clamp mode, by recording optical signals intrinsic to the tissue, imaging signals of voltage sensitive and ion concentration selective compounds, by recording the movement of potassium ions with ion selective electrodes or by recording population responses with extracellular electrodes.

The present invention also provides a compound identifiable by the method of the invention for use in the treatment or prevention of schizophrenia, epilepsy, spike time dependent plasticity, or influencing learning and memory. The above listed diseases, disorders or functions are not intended to limit the applicability of the invention in any way, but are only exemplary. Obviously the present invention will be also useful in other similar conditions, either known today or identified in the future.

It will be understood by a person skilled in the art that the essence of the activity of the compounds identified by the methods of the invention is the modulation of the activity of the AACs, which effect can also be accomplished by a specific nucleic acid vector. Therefore, the present invention also provides a nucleic acid vector capable of modulating the activity of axo-axonic cells by means of expressing a compound that has an effect according to the invention on the axo-axonic cell, for use in the treatment or prevention of schizophrenia, epilepsy, spike time dependent plasticity, or influencing learning and memory. In other embodiments, the compounds can be incorporated into different compositions, which are obvious for the person skilled in the art. Materials and methods for handling and using expression vectors are well known for the person skilled in the respective art, and many well known textbooks can be referenced for detailed teachings [Maniatis et al., Ausubel et al.].

In a further embodiment, the present invention provides the use of the compound according to the invention for the treatment or prevention of schizophrenia, epilepsy, spike time dependent plasticity, or for influencing learning and memory in a mammal, comprising modulating the activity of axo-axonic cells in the said mammal thereby synchronizing the responses of cells postsynaptic to said axo-axonic cells.

Additionally, in a further embodiment, the present invention provides the use of the compound according to the invention in the preparation of a medicament for the treatment or prevention of schizophrenia, epilepsy, spike time dependent plasticity, or for influencing learning and memory in a mammal, comprising modulating the activity of axo-axonic cells in the said mammal thereby synchronizing the responses of cells postsynaptic to said axo-axonic cells.

In a further embodiment, the present invention provides a method for the treatment or prevention of schizophrenia, epilepsy, spike time dependent plasticity, or for influencing learning and memory in a mammal, comprising modulating the activity of axo-axonic cells in the said mammal thereby synchronizing the responses of cells postsynaptic to said axo-axonic cells.

The present invention identifies a novel mechanism in neural input to output coupling through unitary GABAergic synapses from axo-axonic to pyramidal neurons. High sodium channel density and/or sodium channels with a shifted voltage dependence at relatively proximal parts of the axon [Stuart et al. (1997); Colbert and Pan (2002)] and the high density synaptic input in a very high surface/volume ratio area favour the extremely high spike triggering effectiveness of depolarizing inputs from AACs. The unique proximity of input from AACs to the first axonal branch point suggested as the site of spike initiation in neurons might also contribute to postsynaptic activation [Clark et al. (2005)]. In contrast, unitary glutamatergic synapses or de-polarizing GABAergic responses, arriving to the dendritic spines and shafts, have a relatively limited efficacy in triggering axonal action potentials in the postsynaptic cells due to interaction with intrinsic electrotonic properties and other excitatory and inhibitory inputs [London et al. (2002)].

The measured reversal potential of AAC evoked GABAergic responses falls into the subthreshold membrane potential range recorded in cortical neurons. The ionic equilibrium for GABAergic inputs is dynamically regulated by cellular and network mechanisms [Misgeld et al. (1986); Staley et al. (1995); Payne et al. (2003)]. Intraneuronal chloride gradients could be important in neural computation [Gavrikov et al. (2003)] and depolarized GABA reversal potentials in interneurons might lead to physiological GABAergic excitation within networks of GABAergic cells [Martina et al. (2001); Chavas and Marty (2003)]. The membrane potential of pyramidal cells and thus the effectiveness of axo-axonic spike transmission could be regulated by local circuits through dynamic changes in the proportional balance of in excitation and inhibition during diverse cortical tasks [Steriade et al. (1993); Shu et al. (2003)]. Our results paradoxically suggest that postsynaptic hyperpolarization and sodium channel de-inactivation helps axo-axonic spike triggering, but depolarization and sodium channel inactivation could render axo-axonic inputs to shunting or hyperpolarizing. AACs, therefore, might have a dual role in neural circuits. Indeed, firing of AACs stereotypically precedes or follows activation of pyramidal cells depending on the operational state of the network in vivo [Klausberger et al. (2003); Zhu et al. (2004)]. Simultaneous activation of a fraction of the several hundred postsynaptic pyramids innervated by a single AAC [Freund and Buzsaki (1996); Somogyi et al. (1998)] could lead to synchronous recruitment of network activity as observed during the onset of cortical ripples [Klausberger et al. (2003)]. AACs do not target GABAergic interneurons [Freund and Buzsaki (1996); Somogyi et al. (1998)], therefore they are well suited for initiating repeatable event sequences in the cortical microcircuit [Abeles (1991); Ikegaya et al. (2004)] through selective spike triggering in pyramidal cells followed by downstream recruitment of inhibition enforcing spatiotemporal fidelity in signal propagation [Pouille and Scanziani (2004); Traub et al. (1996)].

The present invention is further illustrated by the experimental examples described below, however, the scope of the invention will by no means be limited to the specific embodiments described in the examples.

### BRIEF DESCRIPTION OF THE DRAWINGS

FIG. 1. Compartmentalized reversal potentials for GABAergic inputs associated with differential subcellular distribution of the potassium-chloride cotransporter KCC2. Unitary interactions from basket and AACs to pyramidal cells reveal different reversal potentials for perisomatic and axonal GABAergic inputs (−72 and −54 mV, respectively).

FIG. 2. A, Reconstruction of an AAC in the layer 2/3 of somatosensory cortex of the rat (soma and dendrites: gray, axons: black). B, Light micrographs showing axonal cartridges or candles characteristic to AACs recorded in the rat and human cortex.

FIG. 3. A, Pressure application of GABA to the soma and to the axon initial segment of the same pyramidal cell recorded in perforated patch clamp mode confirmed that the reversal potentials were different in the soma and in the axon initial segment. B, Application of GABA to the axon initial segment of a pyramidal cell recorded with the high [Cl⁻]ᵢₙ intracellular solution in whole cell mode. Spikes with threshold potentials (−64.2±2.8 mV) more hyperpolarized (p<0.007) than the threshold determined with somatically delivered current pulses (−43.1±2.9 mV) were triggered only occasionally (7±3% of trials below somatic threshold potential, n=4 cells). Importantly, pressure application of GABA was not effective in triggering short latency spikes from hyperpolarized membrane potentials unlike synaptic input to the axon initial segment recorded with the same high [Cl⁻]ᵢₙ intracellular solution (FIG. 2B) or with axonal [Cl⁻]ᵢₙ (FIG. 2A). Thus, pressure application of GABA could not mimic the timing and efficacy of spike triggering achieved by synaptically released GABA targeting the axon.

FIG. 4. Localization of KCC2 immunoreactivity in layer 2/3 of rat (top) and human (bottom) cerebral cortex. Gold particles labelling KCC2 (arrows) are pre-dominantly found along somatic (s), dendritic (d) membranes and cytoplasm (arrowheads). Gold particles (double arrows) are also attached to the membrane of the axon hillock (h), but the density of gold particles (double arrowheads) drops on the axon initial segment (AIS). Inset, Quantitative evaluation of immunogold distribution of KCC2 on different subcellular compartments of cortical pyramidal cells. Bars indicate significant differences.

FIG. 5. A model of two compartments corresponding to the soma and the axon initial segment with KCC2 concentrations determined by immunolocalization. We varied the parameters of the diffusion constant of Cl⁻ in the cytoplasm (D) and the maximal unitary turnover rate of KCC2 (vmax). The gray boxes correspond to the range of KCC2 concentration ratios detected by quantitative immunocytochemistry in individual subjects and to the area of postsynaptic axon initial segments innervated by axo-axonic cells in our sample. The [Cl⁻]ᵢₙ gradient detected between the soma and the axon initial segment by our perforated patch recording can be maintained with realistic parameters.

FIG. 6. Spike to spike coupling between axo-axonic cells and pyramidal cells in the cortex. Quadruple recording of an AAC (top, gray) and three pyramidal cells (middle, black pyr 2-4) recorded with axonal [Cl⁻]ᵢₙ in whole cell mode. Single action potentials in the AAC elicited subthreshold depolarizing GABAergic postsynaptic potentials (dIPSPs) on pyramid 2, triggered postsynaptic action potentials in the majority of trials on pyramid 3 with powerful trigger IPSPs (tIPSPs) and initiated disynaptic EPSPs on pyramid 4. Bottom, Schematic diagram of the connections.

FIG. 7. Differential spike triggering efficacy of depolarizing GABA responses arriving to the perisomatic region and to the axon of postsynaptic pyramidal cells. Action potentials in a basket cell and AAC (top), 20 consecutive postsynaptic responses in a pyramidal cell recorded at different membrane potentials (middle) and the threshold (arrows) determined by somatic depolarizing current pulses (bottom). Unlike the basket cell, the AAC triggered postsynaptic spikes with a threshold (−70 mV, arrowhead) below the value detected by somatically injected currents.

FIG. 8. GABAergic and glutamatergic event sequences triggered by AACs in the cortical network. Spikes in an AAC (gray triangles) elicited monosynaptic IPSPs which were followed by convergent disynaptic EPSPs in 54% of trials in a pyramidal cell (black, averages of 30 traces). Application of the GABAA receptor antagonist gabazine (20 μM) blocked the IPSPs as well as the EPSP. Re-patching the presynaptic cell during washout with a different recording pipette did not alter the IPSP-EPSP sequence and its sensitivity to gabazine.

FIG. 9. Both GABAergic and glutamatergic mechanisms are necessary for reliable triggering of disynaptic EPSPs. Paired recording of an unidentified interneuron (gray) and an AAC (black). In control conditions, the interneuron elicited a unitary IPSP in the AAC, the AAC triggered a disynaptic EPSP in the basket cell. Application of the GABAA receptor antagonist gabazine (20 μM) blocked both responses (averages of 20 traces) with a recovery upon washout. The AMPA-receptor antagonist NBQX (10 μM) reversibly eliminated the disynaptic EPSP and, as expected, had no effect on the IPSP.

FIG. 10. The first recording from a human axo-axonic cell shows recurrent, disynaptic EPSPs (arrow) on single consecutive sweeps following the action potential which elicited disynaptic, trigger EPSPs (tEPSPs) capable of initiating third order spikes in a simultaneously recorded interneuron. Disynaptic EPSPs and downstream spikes were blocked by gabazine (20 μM).

FIG. 11. Disynaptic EPSPs or depolarizing afterpotentials (arrows) frequently follow action potentials elicited by current injections in AACs (top) and occasional spike doublets occur (double arrowheads) during spontaneous firing (bottom). Follower spikes in doublets are equivalent to third order spikes in a recurrent circuit.

FIG. 12. The action of AACs is preserved when applying multiple presynaptic action potentials. This example shows action potentials delivered at 60 ms interval in an AAC. Depolarizing afterpotentials (arrows) are similar to excitatory postsynaptic potentials (EPSPs) due to their variable amplitude and paired pulse depression.

### EXAMPLE 1

### Materials and Methods

Electrophysiology and Analysis

All procedures were performed with the approval of the University of Szeged and according to the Declaration of Helsinki. Wistar rats (P20-35) were anaesthetized by the intraperitoneal injection of ketamine (30 mg/kg) and xylazine (10 mg/kg), and following decapitation, coronal slices (350 μm thick) were prepared from the somatosensory cortex.

Human slices were derived from material which had to be removed to gain access for the surgical treatment of basal meningeomas from the left gyrus temporalis inferior with written informed consent of the patients (aged 53 and 58 years) prior to surgery. Anaesthesia was induced with intravenous midazolam and fentanyl (0.03 mg/kg and 1-2 μg/kg, respectively). A bolus dose of propofol (1-2 mg/kg) was administered intravenously. To facilitate endotracheal intubation, the patient recieved 0.5 mg/kg rocuronium. After 120 seconds, the trachea was intubated and the patient was ventilated with a mixture of O₂—N₂O at a ratio of 1:2. Anaesthesia was maintained with sevoflurane at MAC volume of 1.2-1.5. Slices were incubated at room temperature for 1 hour in a solution composed of 130 mM NaCl, 3.5 mM KCl, 1 mM NaH₂PO₄, 24 mM NaHCO₃, 1 mM CaCl₂, 3 mM MgSO₄ and 10 mM D(+)-glucose, saturated with 95% O₂ and 5% CO₂. The solution used during recordings differed only in that it contained 3 mM CaCl₂ and 1.5 mM MgSO₄. Recordings were obtained at ˜35° C. from up to four concomitantly recorded cells visualized in layer 2/3 by infrared differential interference contrast videomicroscopy (Olympus BX60WI microscope, Hamamatsu CCD camera, Luigs & Neumann Infrapatch set-up and two HEKA EPC 10/double patch-clamp amplifiers). Signals were filtered at 8 kHz, digitized at 16 kHz and analyzed with PULSE software (HEKA). Micropipettes (5-7 MW) were filled with three types of solutions. The low [Cl⁻]ᵢₙ solution contained 126 mM K-gluconate, 4 mM KCl, 4 mM ATP-Mg, 0.3 mM GTP-Na₂, 10 mM HEPES, 10 mM phosphocreatine and 8 mM biocytin (pH 7.20; 300 mOsm). The axonal and high [Cl⁻]ᵢₙ solution was different only in K-gluconate (113.5 mM and 42 mM, respectively) and KCl concentrations (16.5 mM and 88 mM, respectively). For perforated patch experiments, 3 mg/ml gramicidin (Sigma) was used, dissolved in DMSO and diluted to a final concentration of 9 μg/ml in the low [Cl⁻]ᵢₙ pipette solution. Access resistance was continuously monitored throughout perforated patch experiments to check membrane rupture. Presynaptic cells were stimulated with brief (2 ms) suprathreshold pulses delivered at >7 s intervals, to minimize intertrial variability. Membrane potentials were corrected for junction potentials. The calculations for [Cl⁻]ᵢₙ were based on the Goldman-Hodgkin-Katz-equation [Kaila et al. (1993)] and assuming a HCO₃⁻/Cl⁻ permeability ratio of 0.2 and an intracellular pH of ˜7.2 ([HCO₃]ᵢₙ, ˜16 mM). During pressure application of GABA (1 mM; 10 kPa, 30 ms), excitatory transmission and GABA_{B} receptors were blocked (20 μM NBQX, 50 μM APV, 60 μM CGP35348). Pressure waves (˜5 μm in diameter) and pipette positions on the soma and along the axon initial segment (30-40 μm from the soma) were imaged and the postsynaptic cell membrane was ruptured by gentle suction pulses to allow biocytin labelling and post-hoc comparison of pipette positions with light microscopic reconstructions. Cells were excluded from analysis if basal dendrites were closer than ˜30 μm to the pressure pipette and/or initial segment. Traces shown are averages or single sweeps of 50-100 consecutive sweeps. Mann-Whitney U-test was used to compare datasets, differences were accepted as significant if p<0.05.

Histology

Visualization of biocytin and correlated light- and electron microscopy was performed as described [Buhl et al. (1994)]. Three-dimensional light microscopic re-constructions were carried out using Neurolucida (MicroBrightfield, Colchester, Vt.) with 100× objective.

Pre-Embedding Immunocytochemistry and Quantitative Analysis

Male Wistar rats (n=3) were deeply anaesthetized as detailed above for electrophysiological experiments. The animals were perfused first with physiological saline for 1 minute, then with ice cold 0.1 M sodium acetate buffer (pH=6.0) containing 2% paraformaldehyde and 0.1% glutaraldehyde for 2 minutes followed by 1 hour fixation with 2% paraformaledyde and 0.1% glutaraledhyde in 0.1 M sodium borate buffer (pH=8.0). Brains were left in the skull at 4° C. overnight, then removed and cut on a vibratome.

Human cortical tissue was derived from material which had to be removed for the surgical treatment of a tumor metastasis located beneath the right gyrus frontalis superior with a written consent of the patient (aged 32 years) prior to surgery. Anaesthesia was performed as described above. The human block of tissue was immersed into ice cold ACSF in the operating theatre and after 20 minutes it was immersed into a solution identical to what was used for the fixation of rat cortex and fixed overnight. Coronal sections (60 μm) were collected in 0.1 M phosphate buffer (PB), treated with 1% sodium borohydrate in 0.1 M PB for 15 mins then washed several times in 0.1 M PB.

To enhance antibody penetration, sections were freeze-thawed in liquid nitrogen. After extensive washes in Tris-buffered saline (TBS, pH=7.4), selected sections were blocked in 10% normal goat serum (NGS) for one hour, then incubated overnight in 2% NGS containing rabbit anti-KCC2 antibody (1:250; Upstate) at room temperature. Following several washes in TBS, sections were incubated in secondary antibody (0.8 nm gold-conjugated goat anti-rabbit IgG; 1:50; Amersham) diluted in TBS containing 0.8% bovine serum albumine (BSA) and 0.1% cold water fish skin gelatine (Aurion) overnight, postfixed in 1% glutaraldehyde in TBS. The ultrasmall gold particles were silver-intensified with R-gent silver intensification kit (Aurion). The sections were treated with 0.5% OsO₄ in 0.1 M PB for 25 minutes at 4° C., contrasted in 1% uranil acetate for 40 minutes and dehydrated in graded alcohol series and embedded in Durcupan. Blocks containing somatosensory cortex were re-embedded and 80 nm thick ultrathin sections were cut with an ultramicrotome (RMC MT-XL) and the ribbons of sections were collected on grids and examined with a Tecnai-12 (FEI) electron microscope. Images were taken by a CCD camera (MegaView III, Soft Imaging System). All images were taken in a depth of <3 μm from the tissue surface, and special care was taken to investigate profiles of axon initial segments and somata from the same depth. The specificity of the anti-KCC2 antibody was extensively studied by the laboratory of origin [Williams et al. (1999)] and was successfully used previously for detecting KCC2 expression pattern in different CNS areas by several laboratories [Rivera et al. (1999); Gulacsi et al. (2003)]. When the application of the primary antibody was omitted from the protocol described above, no specific signal could be detected. For quantification, we first calculated the non-specific labelling density over axon terminals which should not contain KCC2. Then we compared this background to KCC2 immunogold density on somatic and axonal compartments of pyramidal cells using ANOVA with Tukey's correction.

### EXAMPLE 2

### Hyperpolarized Reversal Potential of Axo-Axonic Inputs

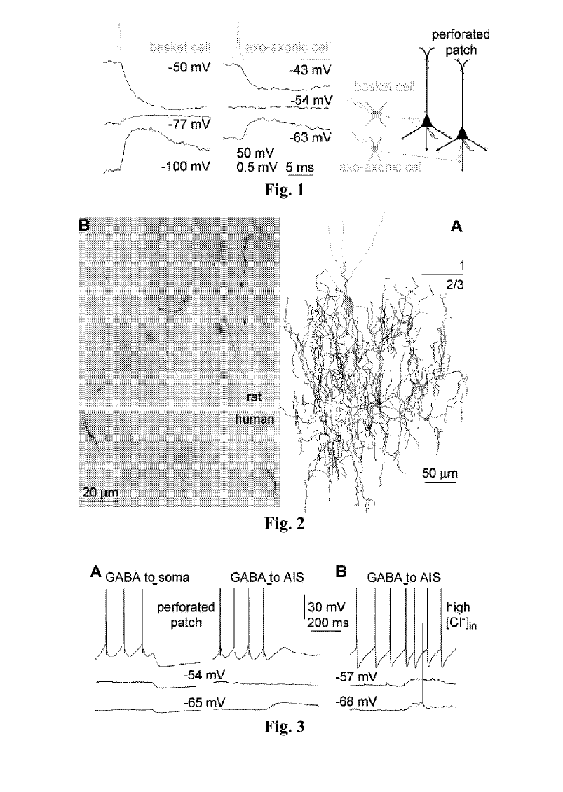

To characterize the interaction of axo-axonic cells (AACs) and their postsynaptic neurons, first the reversal potential of GABAergic inputs arriving to the perisomatic region of pyramidal cells was determined, elicited by AACs and basket cells in layers 2/3 of rat somatosensory cortex in vitro (FIG. 1). Paired recordings of presynaptic interneurons and postsynaptic pyramidal cells were performed using the gramicidin perforated patch placed onto the axon hillock between the soma and the initial segment. Basket cells (n=5), targeting somata (23±7%), dendritic shafts (62±12%) and spines (15±8%) as examined in the electron microscope, elicited unitary IPSPs with reversal potentials of −73.3±3.0 mV. AACs were identified based on light microscopic reconstructions and analysis showing axonal candles or cartridges specific to this cell type [Szentagothai and Arbib (1974); Somogyi (1977)] (FIG. 2). The reversal potential of GABAergic responses triggered by AACs was more depolarized (n=7; −51.1±5.4 mV; p<0.001). Pressure application of GABA to the soma and to the axon initial segment of the same pyramidal cell (n=7) also showed that the reversal potentials were different (−71.2±2.7 mV and −55.1±4.7 mV, respectively, p<0.002, FIG. 3) supporting earlier experiments [Connors et al. (1988)] and our paired recordings. The calculated [Cl⁻]ᵢₙ corresponding to the pooled responses were 16.9±7.6 mM in the axon and 5.9±1.7 mM in the soma [Kaila at al. (1993)].

### EXAMPLE 3

### Differential Subcellular Distribution of KCC2

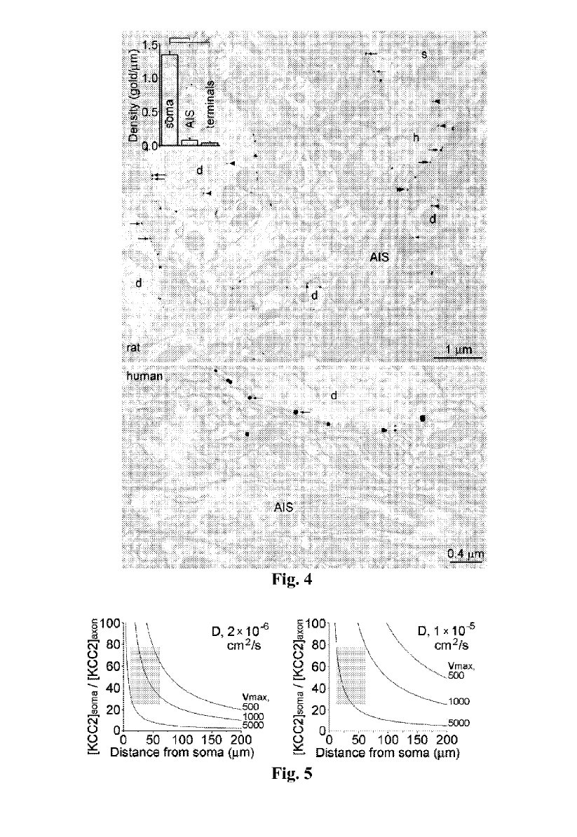

To determine the subcellular distribution of KCC2 on layer 2/3 pyramidal cells of rat and human cortex, high-resolution immunolocalization experiments were performed (FIG. 4). In the rat, membranes of somata (n=38) and membranes of axon initial segments (n=11) contained higher densities of gold particles compared to the background which was measured over nerve terminal membranes (p<0.0001 for soma, p<0.05 for axon, FIG. 4). Comparison of immunogold densities after the subtraction of background labelling (0.04±0.01 gold/μm) showed a ˜44 fold decrease from somatic to axon initial segment plasma membranes (from 1.34±0.04 to 0.08±0.03 gold/μm, p<0.0001). Density levels dropped at the border between the hillock and the initial segment and remained stationary on individual axon initial segments measured up to 40 μm from the soma (FIG. 4). These results on the subcellular distribution of KCC2 were also confirmed in the cortex of a human patient where a ˜52 fold difference in labelling was detected between the membranes of somata (n=20, 1.51 gold/μm) and axon initial segments (n=4, 0.04 gold/μm) after the subtraction of background (0.01 gold/μm).

Although dendrites clearly contained KCC2 labelling (FIG. 4), the detailed quantification of KCC2 levels along the dendritic tree in relation to GABAergic response polarity requires a separate study following reports that the polarity of dendritic IPSPs could be a function of distance from the soma [Connors et al. (1988); Misgeld et al. (1986); Alger and Nicoll (1979), Gulledge and Stuart (2003)]. Thus, GABAergic inputs arriving from basket cells are surrounded by a high concentration of KCC2 setting the reversal potentials at hyperpolarized values. Low KCC2 density and decreased chloride efflux in axon initial segments could support higher [Cl⁻]ᵢₙ leading to depolarizing effects of AACs at physiological subthreshold conditions. To investigate this theory, a model of two compartments corresponding to the soma and the axon initial segment with KCC2 concentrations determined by immunolocalization was applied.

The equations describing the component balance for the amount of Cl⁻ in the soma (1) and in the axon (2) at the steady state can be given as

$$\frac{77 \% {qf}_{1}}{F} - \frac{{v}_{max} {n}_{KCC , 1} {c}_{1}}{{c}_{1} + {K}_{M}} + \frac{DA}{δ} ( {c}_{2} - {c}_{1} ) = 0$$

$$\frac{77 \% {qf}_{2}}{F} - \frac{{v}_{max} {n}_{KCC , 1} {c}_{2}}{{c}_{2} + {K}_{M}} - \frac{DA}{δ} ( {c}_{2} - {c}_{1} ) = 0$$

where the first term is the flow term of Cl⁻ through GABAergic synapses, the second term is the removal of Cl⁻ by the KCC, and the third term is the diffusion between the soma and the axon. The charge through the synapse is q, F is the Faraday-constant, A is the cross sectional area of the axon, δ is the diffusion length, and c₂ and c₁ are the concentrations of Cl⁻ in the axon and in the soma, respectively.

Simplifying the equations by dividing them by vₘₐₓn_{KCC,1} and introducing the new parameters of Q=77% q/(Fvₘₐₓn_{KCC,1}), n_{2/1}=n_{KCC,2}/n_{KCC,1} and D′=DA/δvₘₐₓn_{KCC,1} leads

to

$${Qf}_{1} - \frac{{c}_{1}}{{c}_{1} + {K}_{M}} + {D}^{′} ( {c}_{2} - {c}_{1} ) = 0$$

$${Qf}_{2} - \frac{{n}_{2 / 1} {c}_{1}}{{c}_{2} + {K}_{M}} - {D}^{′} ( {c}_{2} - {c}_{1} ) = 0$$

There are two unknown parameters (Q and D′) in the equations from which Q can be eliminated and the rearrangement of the equations yields

$${D}^{′} = \frac{\frac{{c}_{1} {f}_{2}}{{c}_{1} + {K}_{M}} - \frac{{n}_{2 / 1} {c}_{2} {f}_{1}}{{c}_{2} + {K}_{M}}}{( {f}_{1} + {f}_{2} ) ( {c}_{2} - {c}_{1} )}$$

where f₁=520 s⁻¹, f₂=52 s⁻¹ according to in vivo firing rates of basket cells and AAC during theta oscillations [Klausberger et al. (2003)] and considering a 10 to 1 ratio of basket vs. axo-axonic synapses according to [Farinas and DeFelipe (1991a); Farinas and DeFelipe (1991b)], c₁=6 mM, c₂=17 mM based on our perforated patch recordings of unitary perisomatic and axo-axonic inputs, K_{M}=6.2 mM randomly chosen from the range of 5-10 mM suggested by J. A. Payne (University of California, Davis, personal communication), and the ratio of the amount of KCC in the axon with respect to the soma (n_{KCC,2}/n_{KCC,1}) varies according to the results of our quantitative immunocytochemistry data multiplied by the surface ratio of the soma (a sphere of 20 μm diameter) and axon initial segment (a cylinder with a diameter of 1 μm and a length of 50 μm).

D′ can also be expressed as

$${D}^{′} = \frac{DA}{δ {v}_{max} {n}_{KCC , 1}}$$

where D is the diffusion constant of Cl⁻ in the cytoplasm, A is the is the cross sectional area of the axon, and δ is the length of diffusion along the axon needed to maintain steady state of different Cl⁻ concentrations in the axon and the soma, vₘₐₓ is the maximal unitary turnover rate of KCC2 (>1000 according to J. A. Payne). The parameters of D, n_{KCC,2}/n_{KCC,1} and vₘₐₓ were varied and it was concluded that the [Cl⁻]ᵢₙ gradient detected between the soma and the axon initial segment by the perforated patch recording by the present inventors can be maintained with realistic constraints on [Cl⁻] diffusion and KCC2 activity (see also FIG. 5).

### EXAMPLE 4

### Spike Transmission from Axo-Axonic Cells to Pyramidal Cells

Based on the results presented above, the effect of AACs (n=22) on pyramidal cells (n=81) held at the resting membrane potential (−72±2 mV) were tested and recorded in whole cell mode with axonal [Cl⁻]ᵢₙ determined in perforated patch experiments (FIG. 6). The majority (59.2%, n=48) of pyramidal cells did not receive input initiated by AACs and the rest of pyramidal cells showed three types of responses to single presynaptic action potentials.

Subthreshold depolarizing GABAergic postsynaptic potentials (dIPSPs) which had short, monosynaptic onset latencies (0.69±0.17 ms) and amplitudes of 0.61±0.42 mV were elicited in 24 pyramidal cells (29.6%).

In the second group of pyramidal cells (n=3, 3.7%), AACs (n=3) triggered postsynaptic action potentials through powerful unitary GABAergic postsynaptic potentials or “trigger IPSPs” (tIPSPs). Single spikes in a presynaptic AAC elicited postsynaptic action potentials with a probability of 60±7%, which had peak latencies of 2.73±0.24 ms on average and a temporal variance of 0.48±0.17 ms in individual connections. The amplitudes of subthreshold tIPSPs (3.62±0.41 mV) were larger than dIPSPs not capable of triggering postsynaptic spikes at all (p<0.02).

The third type of response was observed in 6 pyramidal cells (7.4%) in which AACs (n=5) initiated depolarising responses with onset latencies longer than that of the onset of dIPSPs or the peak of postsynaptic action potentials triggered by tIPSPs (3.48±0.31 ms; p<0.001 and p<0.02, respectively). The latency difference between tIPSP evoked spikes and the long latency depolarising responses (˜0.85 ms) was similar to the delay of monosynaptic PSPs, suggesting that these long latency responses correspond to disynaptic EPSPs elicited by unrecorded pyramidal cell(s). Moreover, disynaptic EPSP-like events occurred with probabilities (67±14%) similar to that of tIPSP triggered spikes. Two axo-axonic cells elicited each of the three types of postsynaptic responses diverging onto different postsynaptic pyramidal cells (FIG. 6).

The above testing was also repeated by applying multiple presynaptic action potentials instead of a single stimulus. This experiment showed that action potentials delivered at 60 ms interval in an AAC resulted in depolarizing afterpotentials similar to excitatory postsynaptic potentials (FIG. 12).

### EXAMPLE 5

### Efficacy of Somatic and Axonal Excitatory Inputs

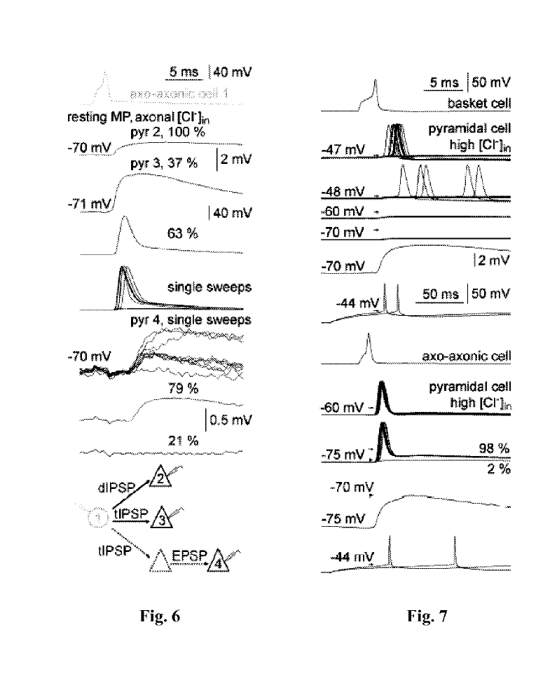

To uncover the mechanisms underlying AAC evoked postsynaptic action potentials, the spike triggering efficacy of depolarizing GABAergic synapses arriving to the soma/proximal dendrites and the axon of postsynaptic cells were compared. The effect of basket cells and AACs was measured during whole-cell recordings from the postsynaptic pyramidal cells with intracellular solutions setting both types of GABAergic responses to depolarizing (10.33±4.57 mV and 8.31±4.42 mV respectively, measured at −68.7±5.6 mV resting membrane potential). While evoking single presynaptic spikes, the membrane potential of the postsynaptic cells was varied to determine the threshold for unitary input triggered action potentials (FIG. 7). For comparison, threshold potentials were also determined with current pulses through somatically placed electrodes (FIG. 7). The postsynaptic spike threshold was similar in response to somatic current pulses (−40.9±4.2 mV, n=14) and inputs evoked by basket cells (p<0.242; −43.9±4.7 mV, n=10) making basket cells incapable of spike triggering from resting membrane potential. In contrast, the threshold for postsynaptic spikes triggered by AACs was significantly more hyperpolarized (p<0.005, −65.3±5.5 mV, n=4) than the somatic spike threshold corroborating our experiments using axonal [Cl⁻]ᵢₙ and in line with reports showing that the axon has lower threshold for spike initiation than the somatodendritic domain in pyramidal cells [Stuart et al. (1997); Colbert and Pan (2002)]. Accordingly, AACs not only triggered spikes from the resting membrane potential, but also elicited action potentials with high efficacy (83.2±22.8%). Moreover, axo-axonic inputs recruited postsynaptic action potentials with a shorter latency (1.857±0.316 ms and 3.281±1.155 ms, p<0.001) and smaller temporal variance (0.168±0.094 ms and 0.887±0.695 ms, p<0.001) than inputs elicited by basket cells. T hus, hyperpolarized axonal threshold for action potential initiation boosts the efficacy of depolarizing inputs and contributes to axo-axonic triggering of spikes in pyramidal cells.

### EXAMPLE 6

### Multiple Synaptic Events Triggered by Axo-Axonic Cells

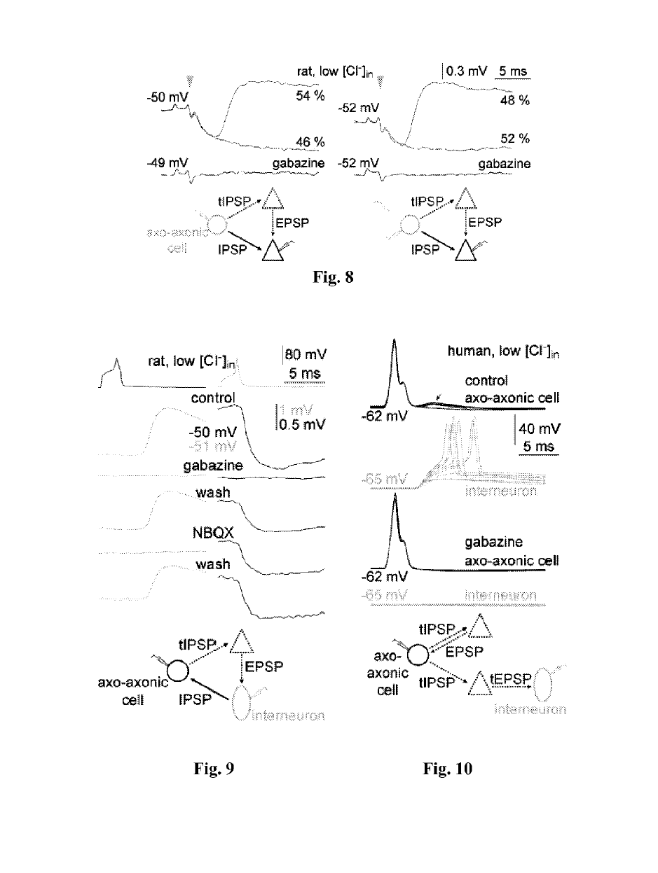

In supragranular cortical layers, pyramidal neurons are the exclusive locally triggered sources of glutamatergic EPSPs and the only targets of AACs [Somogyi et al. (1998)]. To study the network effect of AACs uniquely positioned in the rat and human cortex, simultaneous recordings from up to four neighbouring cortical neurons were applied using an intracellular solution with low [Cl⁻]ᵢₙ in order to easily discriminate excitatory and inhibitory postsynaptic potentials (FIGS. 8-11).

Single spikes in rat AACs triggered temporally fixed series of multiple synaptic events with reliabilities characteristic to unitary, single cell to single cell transmission. AACs in these experiments apparently functioned as if they were inhibitory and excitatory neurons activated in a series (FIG. 8). Seventeen out of 31 AACs (54.8%) elicited short latency (0.81±0.31 ms) unitary IPSPs in pyramidal cells (n=9) which were followed by longer latency (3.71±0.90 ms, p<0.001), presumably disynaptic EPSPs on the same (n=3) or different (n=5) pyramidal cells and on various interneurons (n=19). The disynaptic EPSPs on pyramidal cells and interneurons had onset latencies (3.71±1.12 ms), failure rates (25.8±20.7%) corresponding to tIPSP evoked action potentials. Abandoning and re-patching of the recorded AACs (n=4) with a new pipette did not change the identified synaptic signal sequences excluding the possibility that multiple events could be activated by interactions between the recording pipette and parts of different cells (FIG. 7).

Pharmacological tools were applied and corroborated the results of axonal [Cl⁻]ᵢₙ experiments by showing that AACs are capable of recruiting local glutamatergic cells through GABAergic synapses (n=7, FIG. 8-11). The specific GABA_{A} receptor antagonist gabazine (20 μM) blocked monosynaptic IPSPs as well as disynaptic EPSPs reversibly (FIGS. 8 and 9). As expected, application of the AMPA receptor antagonist NBQX (10 μM) blocked disynaptic EPSPs with a recovery upon washout (FIG. 9). The dual sensitivity of disynaptic EPSPs to gabazine and NBQX rules out the involvement of gap junctions in the underlying circuit.

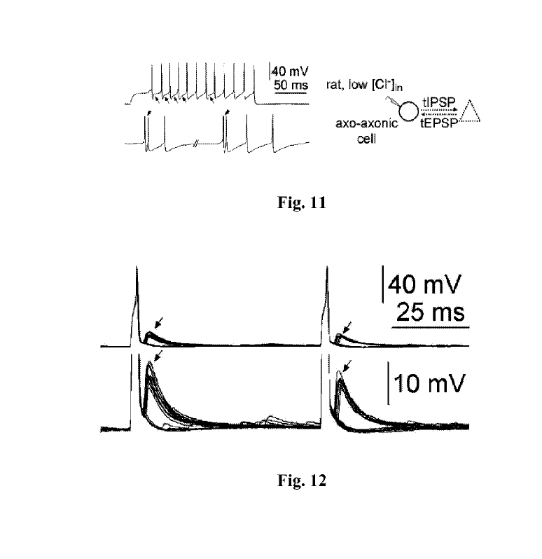

During this experiment, the present inventors performed the first recordings from human layer 2/3 axo-axonic cells (n=2) and confirmed GABAergic recruitment of excitation (FIG. 10). Moreover, a human example also showed that second order spikes in pyramidal cells initiated by axo-axonic GABAergic synapses project powerfully to neighbouring interneurons and trigger third order action potentials further prolonging event sequences downstream of AACs. Third order activation was also detected in AACs; pyramidal cells activated by tIPSPs feed back to axo-axonic cells both in human and rat cortex (FIGS. 10 and 11). These disynaptic, recurrent EPSPs were readily identified while recording the firing pattern of several AACs (n=19, FIG. 11) and were similar to depolarizing afterpotentials found earlier in hippocampal AACs [Buhl et al. (1994)]. In these cells, like in the hippocampal AACs, spike doublets were also identified during spontaneous firing predominantly at the beginning of short action potential trains (FIG. 11). Spike doublets promote the temporal summation of axo-axonic depolarizing responses and might enhance spike to spike transmission.

### EXAMPLE 7

### Testing Compounds Up- or Downregulating the Activity of AACs

Slices are obtained from the somatosensory cortex of Wistar rats (P19-35) and maintained as described [Tamas et al. (2000)]. AACs are identified based on disynaptic EPSPs occurring after their action potentials, their fast spiking firing pattern and their axonal and dendritic morphology [Szentagothai and Arbib (1974); Somogyi (1977)]. Whole-cell patch clamp recordings are carried out at ˜35° C. on AACs as detailed in Example 1 and baseline firing characteristics are recorded.

The brain slices are perfused with the putative active agent to allow equilibrium (approximately for 20 minutes as shown in FIG. 8-10), and signals are recorded with HEKA EPC9/2 amplifiers in fast current clamp or voltage clamp whole cell mode and are filtered at 8 kHz, digitized at 16 kHz and analyzed with PULSE software.

The membrane potential (current clamp), holding current (voltage clamp) and firing characteristics (current clamp) of the AACs are compared to the baseline. Depolarization, increased holding current, enhanced firing characteristic or an increase in the frequency of disynaptic EPSPs suggests that the test compound is a stimulating agent of axo-axonic cells cells. In contrast, if hyperpolarization, decrease in holding current, the decrease of the firing properties and/or a decrease in the frequency of disynaptic EPSPs is detected, the test compound is an inhibitor of axo-axonic cells. In the case of complete blockade or termination of the response of AACs, the test compound effectively kills the function of AACs.

### REFERENCES

Abeles, M.: Corticonics (Cambridge University Press, Cambridge, 1991).
Alger, B. E. & Nicoll, R. A.: GABA-mediated biphasic inhibitory responses in hippocampus. Nature 281, 315-317 (1979).
Arellano, J. I., Munoz, A., Ballesteros-Yanez, I., Sola, R. G. and DeFelipe, J.: Histopathology and reorganization of chandelier cells in the human epileptic sclerotic hippocampus. Brain 127, 45-64 (2004)
Ausubel, P. M. et al.: Current Protocols in Molecular Biology, John Wiley and Sons, Inc., New York, N.Y.
Buhl, E. H. et al.: Physiological properties of anatomically identified axo-axonic cells in the rat hippocampus. J. Neurophysiol. 71, 1289-1307 (1994).
Chavas, J. & Marty, A.: Coexistence of excitatory and inhibitory GABA synapses in the cerebellar interneuron network. J. Neurosci. 23, 2019-2031 (2003).
Clark, B. A., Monsivais, P., Branco, T., London, M. and Hausser, M.: The site of action potential initiation in cerebellar Purkinje neurons. Nat. Neurosci. 8, 137-139 (2005).
Colbert, C. M. and Pan, E.: Ion channel properties underlying axonal action potential initiation in pyramidal neurons. Nat. Neurosci. 5, 533-538 (2002).
Connors, B. W., Malenka, R. C. and Silva, L. R.: Two inhibitory postsynaptic potentials, and GABAA and GABAB receptor-mediated responses in neocortex of rat and cat. J. Physiol. 406, 443-468 (1988).
Dan, Y. and Poo, M. M.: Spike timing-dependent plasticity of neural circuits. Neuron 44, 23-30 (2004)
Farinas, I. and DeFelipe, J.: Patterns of synaptic input on corticocortical and corticothalamic cells in the cat visual cortex. II. The axon initial segment. J. Comp. Neurol. 304, 70-77 (1991).
Farinas, I. and DeFelipe, J.: Patterns of synaptic input on corticocortical and corticothalamic cells in the cat visual cortex. I. The cell body. J. Comp. Neurol. 304, 53-69 (1991).
Freund, T. F. and Buzsaki, G.: Interneurons of the hippocampus. Hippocampus 6, 347-470 (1996).
Gavrikov, K. E., Dmitriev, A. V., Keyser, K. T. and Mangel, S. C.: Cation—chloride cotransporters mediate neural computation in the retina. Proc. Natl. Acad. Sci. USA. 100, 16047-16052 (2003).
Gulacsi, A. et al.: Cell type-specific differences in chloride-regulatory mechanisms and GABA(A) receptor-mediated inhibition in rat substantia nigra. J. Neurosci. 23, 8237-8246 (2003).
Gulledge, A. T. and Stuart, G. J.: Excitatory actions of GABA in the cortex. Neuron 37, 299-309 (2003).
Howard, A., Tamas, G. and Soltesz, I.: Lighting the chandelier: new vistas for axo-axonic cells. Trends Neurosci 28, 310-316 (2005)
Ikegaya, Y. et al.: Synfire chains and cortical songs: temporal modules of cortical activity. Science 304, 559-564 (2004).
Kaila, K., Voipio, J., Paalasmaa, P., Pasternack, M. and Deisz, R. A.: The role of bi-carbonate in GABAA receptor-mediated IPSPs of rat neocortical neurones. J. Physiol. 464, 273-289 (1993).
Klausberger, T. et al.: Brain-state- and cell-type-specific firing of hippocampal interneurons in vivo. Nature 421, 844-848 (2003).
Lewis, D. A., Hashimoto, T. and Volk, D. W.: Cortical inhibitory neurons and schizophrenia. Nat Rev Neurosci 6, 312-324 (2005)
London, M., Schreibman, A., Hausser, M., Larkum, M. E. and Segev, I.: The information efficacy of a synapse. Nat. Neurosci. 5, 332-340 (2002).
Maniatis et al.: Molecular Cloning—A Laboratory Manual; Cold Spring Harbor, (1982)
Martina, M., Royer, S. and Pare, D.: Cell-type-specific GABA responses and chloride homeostasis in the cortex and amygdala. J. Neurophysiol. 86, 2887-2895 (2001).
Misgeld, U., Deisz, R. A., Dodt, H. U. and Lux, H. D.: The role of chloride transport in postsynaptic inhibition of hippocampal neurons. Science 232, 1413-1415 (1986).
Payne, J. A., Rivera, C., Voipio, J. and Kaila, K.: Cation-chloride co-transporters in neuronal communication, development and trauma. Trends Neurosci. 26, 199-206 (2003).
Pouille, F. and Scanziani, M.: Routing of spike series by dynamic circuits in the hippocampus. Nature 429, 717-723 (2004).
Rivera, C. et al.: The K+/Cl− co-transporter KCC2 renders GABA hyperpolarizing during neuronal maturation. Nature 397, 251-255 (1999).
Shu, Y., Hasenstaub, A. and McCormick, D. A.: Turning on and off recurrent balanced cortical activity. Nature 423, 288-293 (2003).
Singer, W. and Gray, C. M.: Visual feature integration and the temporal correlation hypothesis. Annu Rev Neurosci 18, 555-586 (1995)
Somogyi, P.: A specific “axo-axonal” interneuron in the visual cortex of the rat. Brain Res. 136, 345-350 (1977).
Somogyi, P., Tamas, G., Lujan, R. and Buhl, E. H.: Salient features of synaptic organisation in the cerebral cortex. Brain Res. Brain Res. Rev. 26, 113-135 (1998).
Staley, K. J., Soldo, B. L. and Proctor, W. R.: Ionic mechanisms of neuronal excitation by inhibitory GABAA receptors. Science 269, 977-981 (1995).
Steriade, M., Nunez, A. and Amzica, F.: A novel slow (<1 Hz) oscillation of neocortical neurons in vivo: depolarizing and hyperpolarizing components. J. Neurosci. 13, 3252-3265 (1993).
Stuart, G., Spruston, N., Sakmann, B. and Hausser, M.: Action potential initiation and backpropagation in neurons of the mammalian CNS. Trends Neurosci. 20, 125-131 (1997).
Szentagothai, J. and Arbib, M. A.: Conceptual models of neural organization. Neurosci. Res. Prog. Bull. 12, 305-510 (1974).
Tamas, G. et al., Nat. Neurosci. 3, 366 (2000)
Traub, R. D., Whittington, M. A., Stanford, I. M. and Jefferys, J. G.: A mechanism for generation of long-range synchronous fast oscillations in the cortex. Nature 383, 621-624 (1996).
Williams, J. R., Sharp, J. W., Kumari, V. G., Wilson, M. and Payne, J. A.: The neuron-specific K—Cl cotransporter, KCC2. Antibody development and initial characterization of the protein. J. Biol. Chem. 274, 12656-12664 (1999).
Zhu, Y., Stornetta, R. L. and Zhu, J. J.: Chandelier cells control excessive cortical excitation: characteristics of whisker-evoked synaptic responses of layer 2/3 non-pyramidal and pyramidal neurons. J. Neurosci. 24, 5101-5108 (2004).

1. A method for identifying an agent capable of specifically modulating or eliminating the action of axo-axonic cells, comprising
a) identifying an axo-axonic cell connected to the initial segment of the axon (within 30-40 μM from the soma or within the region situated between the soma and the first branchpoint of the axon) of at least one functional postsynaptic pyramid cell in a biological sample, cell culture or live animal,
b) stimulating the said axo-axonic cell,
c) measuring the reversal of postsynaptic potential evoked by the said axo-axonic cell,
d) exposing the said axo-axonic cell to a test compound,
e) repeating step b),
f) repeating step c),
g) identifying the test compound as a specific modulator or blocking agent of axo-axonic cells if the measured reversal of postsynaptic potential in step f) significantly differs from that measured in step c).

2. An experimental kit for performing the method according to claim 1 comprising the following:
a cell culture comprising axo-axonic cells,
a medium comprising instructions—and/or optionally means—for performing the method according to claim 1,
and optionally further comprising
at least one postsynaptic cell connected to an axo-axonic cells,
means and/or instructions for detecting depolarizing postsynaptic potential on the postsynaptic pyramid cell evoked by the said axo-axonic cells,
appropriate buffers and culture media.

3. A compound identifiable by the method of claim 1 for use in the treatment or prevention of schizophrenia, epilepsy, spike time dependent plasticity, or influencing learning and memory.

4. A compound according to claim 3 which is expressed within the axo-axonic cell by a nucleic acid vector.

5. A method for the treatment or prevention of schizophrenia, epilepsy, spike time dependent plasticity, or for influencing learning and memory in a mammal, comprising modulating the activity of axo-axonic cells in the said mammal thereby synchronizing the responses of cells postsynaptic to said axo-axonic cells.
